(19) United States
(12) Patent Application Publication
(10) Pub. No.: US 2004/0194541 A1
(43) Pub. Date: Oct. 7, 2004

(54) High-Q LC circuit moisture sensor

(75) Inventors: Faiz Feisal Sherman, Mason, OH (US); Vladimir Gartstein, Cincinnati, OH (US); Kendal William Kerr, Okeana, OH (US); Jim Allen McCurdy, Liberty Township, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/832,221

(22) Filed: Apr. 26, 2004

Related U.S. Application Data
(63) Continuation-in-part of application No. 10/442,503, filed on May 21, 2003.
(60) Provisional application No. 60/387,474, filed on Jun. 10, 2002.

(51) Int. Cl. A61K 7/06

(52) U.S. Cl. 73/73

(57) ABSTRACT

A device is provided for the measurement of the moisture content of a substrate. The device utilizes a high-Q LC circuit having a resonant frequency. The LC circuit utilizes a high-Q inductor and a capacitor. The device also utilizes a high frequency signal generator, operable to couple power to the capacitor, electrically coupled to the LC circuit and a fiber matrix modification unit. The resonant frequency of the LC circuit is changeable in response to the moisture content of the substrate placed within the fiber matrix modification unit and proximate to the capacitor.

### CROSS REFERENCE TO RELATED APPLICATIONS

This application is a continuation-in-part of U.S. patent application Ser. No. 10/442,503 filed on May 21, 2003, which claims the benefit of U.S. Provisional Application No. 60/387,474, filed Jun. 10, 2002.

### FIELD OF THE INVENTION

The present invention relates generally to measurement sensors and, more particularly, to a sensor for measuring a property of a substrate, such as the internal and external moisture content of biological systems such as hair.

### BACKGROUND OF THE INVENTION

Fibrous substrates such as human hair generally comprise complex proteins called alpha-keratins. Alpha keratin fibers, including wool and hair, have a special affinity for water. Hair is hygroscopic and permeable and can absorb water from the environment. Under normal conditions, water accounts for about 12% to 15% of the composition of hair. Further, hair can absorb more than 30% of its own weight in water. Typically, hair absorbs about 30% of its own weight of water at saturation. If the hair is damaged, this percentage can approach 45%. However the ability of damaged hair to retain water within the hair fibers that gives hair its healthy appearance is reduced. As a result of this interaction with water, it is believed that nearly all physical characteristics of keratinous fibers are modified in the presence of water. Examples include variations in length and diameter, changes in internal viscosity, hair holding and setting properties, hair strength, and electro-optic properties.

Moisture sensing devices have been developed in the past to determine the moisture level in hair, and have relied on various techniques including resistance measurements to obtain the desired indication. However, these methods only work well for a known cross sectional quantity and density of the relatively wet hair being measured. As the hair density, wetness, or compactness is varied, these measurement techniques fail. Additionally, these techniques rely primarily on the moisture content outside of the hair fiber for the measurement, and do not have the ability to accurately measure moisture content within hair fibers as well.

Thus, there is a need for a moisture-sensing device that can accurately, and reliably, determine the moisture content of a substrate, including keratinous fibers such as hair.

### SUMMARY OF THE INVENTION

The present invention provides a device for measuring the moisture content of a substrate. The device comprises a high-Q LC circuit having a resonant frequency. The LC circuit utilizes a high-Q inductor and a capacitor. The device also comprises a high frequency signal generator, operable to couple power to the capacitor, electrically coupled to the LC circuit, and a fiber matrix modification unit. The resonant frequency of the LC circuit is changeable in response to the moisture content of the substrate when the substrate is placed within the fiber matrix modification unit and proximate to the capacitor.

The present invention also provides a circuit for a device capable of measuring the moisture content of a substrate. The circuit comprises a high-Q LC circuit, comprising a high-Q inductor and a capacitor. The circuit has a resonant frequency and a high frequency signal generator electrically coupled thereto. The high frequency signal generator is operable to couple power to the capacitor. The resonant frequency of the LC circuit is changeable in response to the moisture content of the substrate placed proximate to the capacitor.

The present invention further provides a method for measuring the moisture content of a substrate by first providing a high-Q LC circuit having a high-Q inductor and a capacitor and having a shiftable resonance curve. Second, the substrate is introduced proximate to the capacitor. Third, the output of the high-Q LC circuit is measured. Next, the output is compared to a reference to determine the moisture content of the substrate.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the invention.

FIG. 1 is a functional block diagram of a directional coupler sensor in accordance with the principles of the present invention;

FIG. 2A is a circuit representation of a high frequency signal generator for use in the sensor of FIG. 1 in accordance with one embodiment of the present invention;

FIG. 2B is a circuit representation of a directional coupler for use in the sensor of FIG. 1 in accordance with one embodiment of the present invention;

FIG. 2C is a circuit representation of a moisture content detector for use in the sensor of FIG. 1 in accordance with one embodiment of the present invention;

FIG. 2D is a circuit representation of a pressure sensor for use in the sensor of FIG. 1 in accordance with one embodiment of the present invention;

FIG. 2E is a circuit representation of a voltage regulator for use in the sensor of FIG. 1 in accordance with one embodiment of the present invention;

FIG. 3 is a top plan view of the sensor of FIG. 1 shown integrated onto a printed circuit board;

FIG. 3A is a cross-sectional view taken along line 3A-3A of FIG. 3;

FIG. 4 is a perspective view of a directional coupler sensor system in accordance with one embodiment of the present invention;

FIG. 4A is an enlarged front elevational view of a hair clamping device for use in the sensor system of FIG. 4, illustrating the clamping device in an open position to receive hair in the device;

FIG. 4B is a view similar to FIG. 4A, illustrating the clamping device in a closed position to clamp hair in the device;

FIGS. 5A and 5B are side elevational views of a hair brush incorporating the directional coupler sensor of the present invention;

FIG. 6 is a graph illustrating the relationship between output voltage of the directional coupler sensor and relative humidity for various switches of hair;

FIG. 7 is a graph illustrating the relationship between moisture content of hair by weight and relative humidity of hair;

FIG. 8 is a graph illustrating the relationship between output voltage of the directional coupler sensor and pressure applied to pack the hair;

FIG. 9A is a perspective view of an alternative device for the measurement of the moisture content of a substrate;

FIG. 9B is a perspective view of another alternative device for the measurement of the moisture content of a substrate;

FIG. 10 is a functional block diagram of a high resonant, high-Q circuit in accordance with the principles of the present invention; and,

FIGS. 11A, 11B, and 11C are graphic representations of exemplary resonance curves for a high resonant high-Q circuit in an open circuit condition, in the presence of a low moisture substrate, and in the presence of a saturated substrate, respectively.

### DETAILED DESCRIPTION

All documents cited in the Detailed Description of the Invention are, in relevant part, incorporated herein by reference; the citation of any document is not to be construed as an admission that it is prior art with respect to the present invention.

A. Directional Coupler

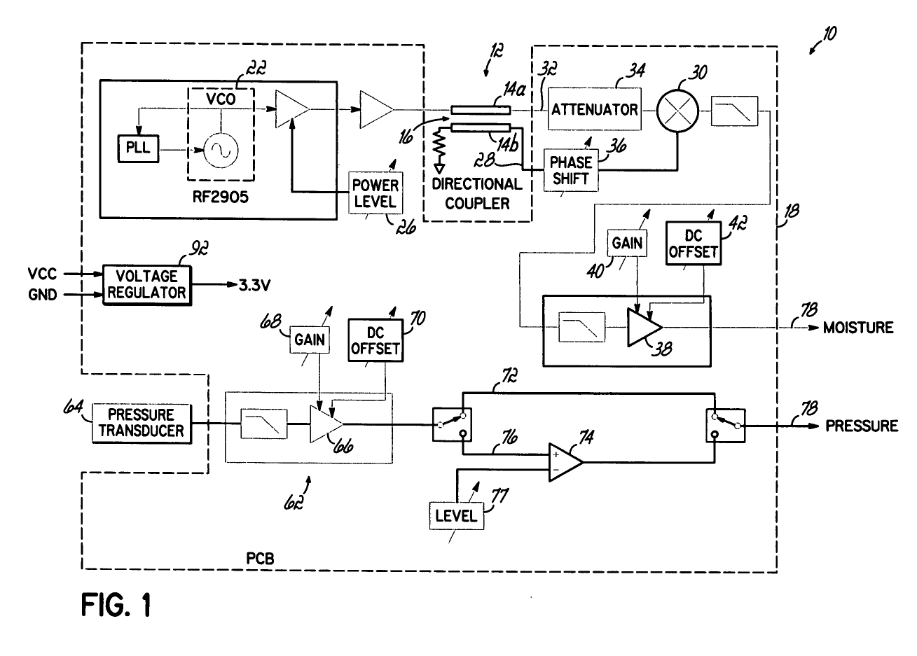

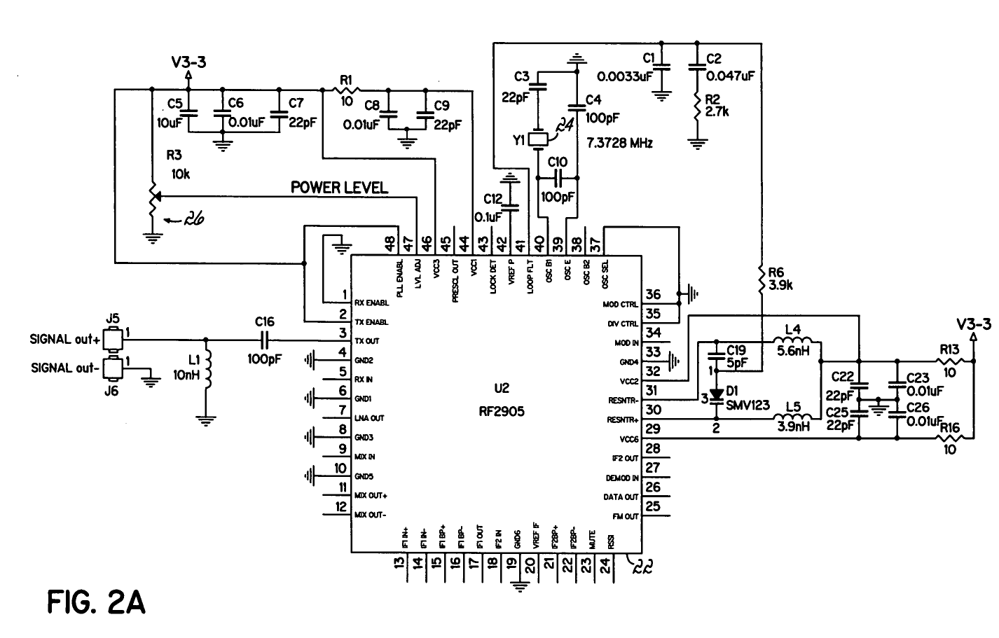

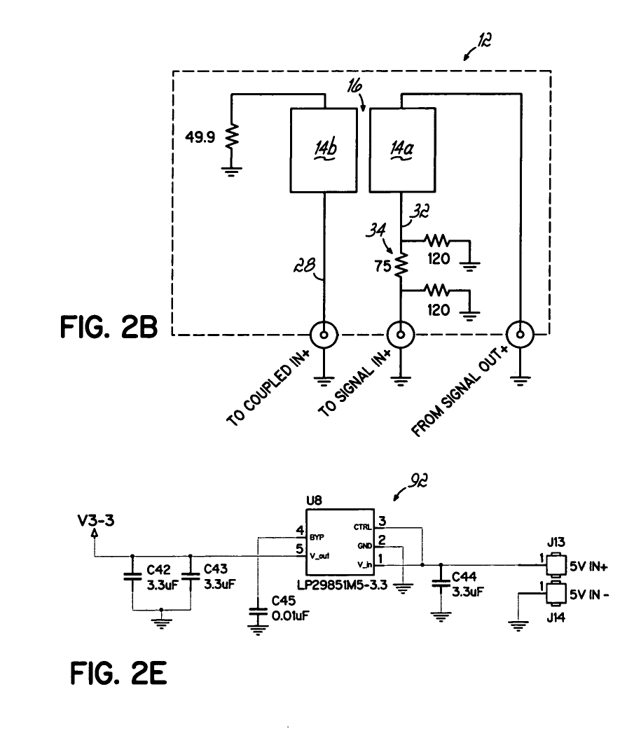

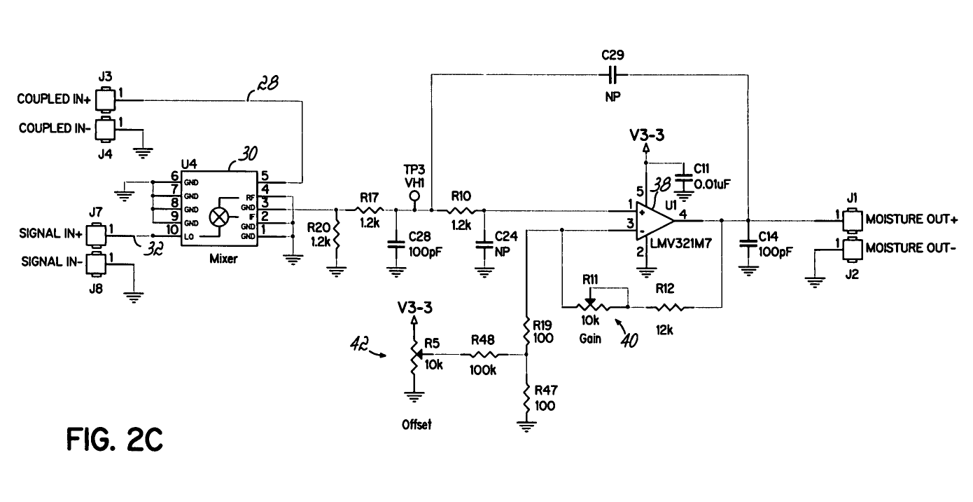

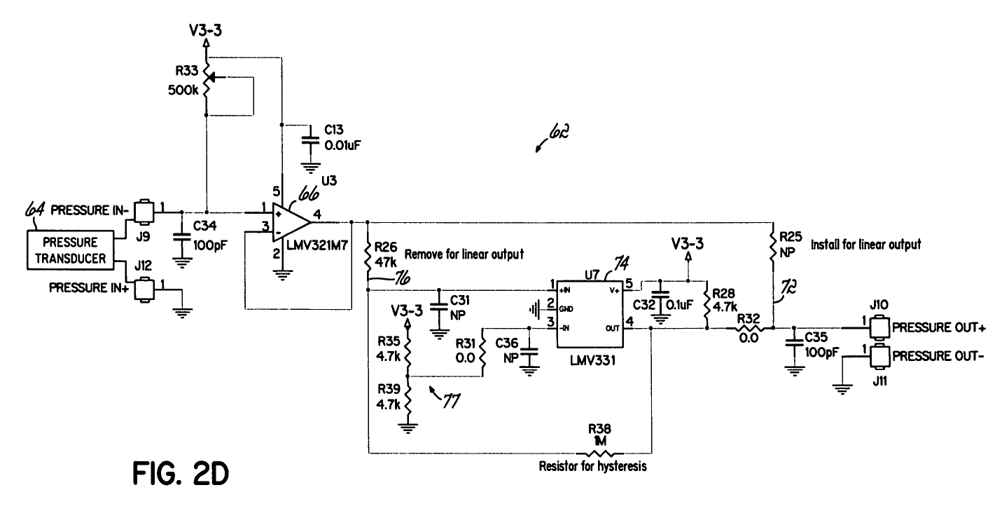

Referring now to the Figures, and to FIGS. 1 and 2A-2E in particular, a directional coupler sensor 10 is shown in accordance with the principles of the present invention. For the sake of simplicity, the sensor 10 will be described herein in connection with measuring the moisture content of hair. However, it will be appreciated by those of ordinary skill in the art that the present invention has use in a wide variety of applications and is therefore not limited to the analysis of hair or the measurement of moisture content in a substrate. Rather, the sensor 10 of the present invention is readily adaptable to analyze a wide variety of substrates, a wide variety of characteristics of these substrates (i.e., chemical and physical properties), and to measure different moisture related properties of those substrates as will be readily appreciated by those of ordinary skill in the art.

For example, in the measurement of the moisture content of a substrate, the sensor 10 of the present invention operates under the principle that as the moisture content of a substrate increases, so does its effective relative electrical impedance. As will be described in greater detail below, the sensor 10 is designed to measure the relative impedance of a substrate, and from that measurement, the moisture content of the substrate can be determined. The moisture content value may be presented on a visual display, indicated through a user-perceptible audible tone and/or used as a control signal to control a function of a device.

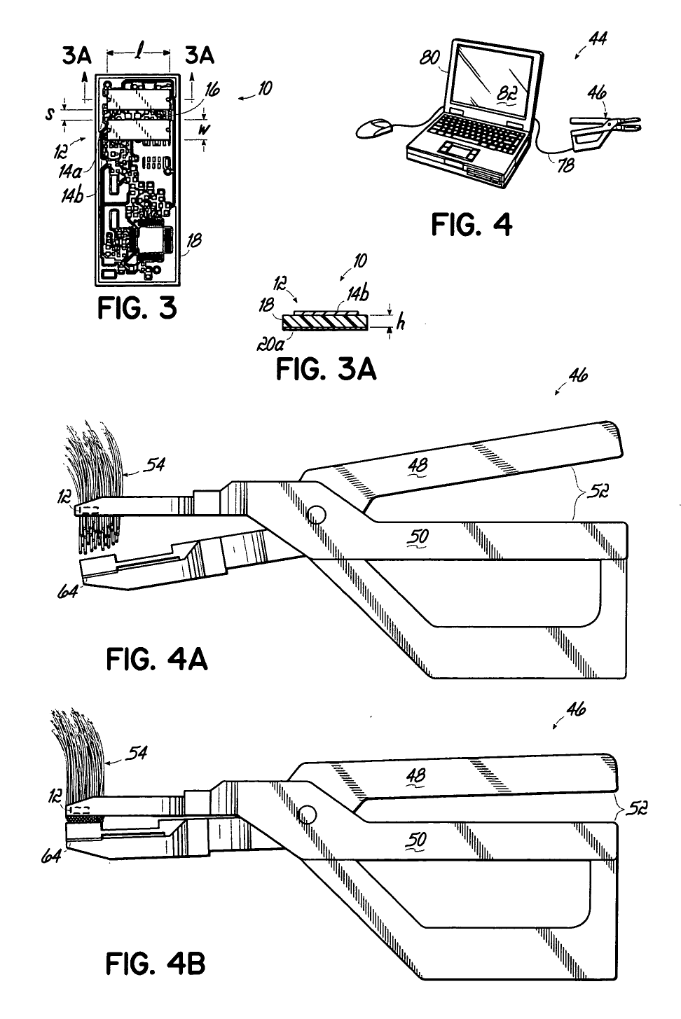

As shown in FIGS. 1, 2A-2E, 3 and 3A, the sensor 10 incorporates a high frequency directional coupler 12 having a pair of generally parallel strips 14a and 14b that define a coupling gap 16 therebetween. In one embodiment of the present invention, the parallel strips 14a, 14b are supported on an FR4 printed circuit board 18 (FIGS. 3 and 3A) having a ground plane 20 formed on a lower surface of the board 18. In one embodiment of the present invention, the height “h” of the PCB 18 is 0.062 in., each strip 14a, 14b has a width “w” of 0.15 in. and a length “1” of 0.350 in., and the coupling gap 16 has a gap distance “s” of 0.020 in. Of course, it will be appreciated by those of ordinary skill in the art that other dimensions of the PCB 18, strips 14a, 14b and gap 16 are possible as well depending on a particular application as will be described in detail below.

A high frequency signal generator 22 is electrically coupled to strip 14a and is operable to generate an electromagnetic field across the coupling gap 16 that couples power to strip 14b with the substrate placed across, i.e., generally normal to the longitudinal axis of, the coupling gap 16 in a packed manner as will be described in detail below. The signal generator 22 generates a coupled power signal in the coupled strip 14b that has an amplitude related to the impedance, and therefore the moisture content, of the substrate placed across the coupling gap 16. The signal generator 22 is phase locked to a crystal reference 24 (FIG. 2A) to maintain frequency and therefore measurement accuracy, stability and repeatability and has an adjustable power 26. The signal generator 22 is preferably operable to generate signals in the VHF to UHF frequency ranges, i.e., between about 30 MHz and about 3 GHz, although other frequency ranges are possible as well. In accordance with one embodiment of the present invention, the signal generator 22 may operate at about 1 GHz, such as frequencies ranging from about 860 MHz to about 928 MHz, and more preferably from about 865 MHz to about 915 MHz, and most preferably about 915 MHz, since it is contemplated that the water content of a substrate may be most accurately determined by its measured impedance in the near GHz range.

In accordance with one aspect of the present invention, the sensor 10 utilizes the reverse power coupling variation of the high frequency directional coupler 12 to measure the change in the impedance of the material placed across the coupling gap 16. As the substrate is packed across the coupling gap 16, the directional coupler 12 becomes mismatched, and this mismatch causes a monotonic increase in the reverse power coupling of the directional coupler 12 as the impedance across the gap 16 is increased as the result of increased moisture content of the material. The amplitude of the reversed power in the reflected power leg 28 (FIGS. 1 and 2B) from strip 14b is generally a direct measure of the impedance, and hence the moisture content, of the substrate placed across the coupling gap 16. As will be described in detail below, the moisture content of the substrate, i.e., its water content by weight, can be determined from the measured impedance of the sample.

Further referring to FIGS. 1 and 2A-2E, the forward power signal from strip 14a is electrically coupled to one port of a mixer 30 through a forward power leg 32 (FIGS. 1 and 2B) and an attenuator 34. For example, the forward power signal may be attenuated to about −10 dBm by the attenuator 34. The coupled power signal from strip 14b is phase shifted by phase shifter 36 and is electrically coupled to another port of the mixer 30 through the reflected power leg 28. The mixer 30 may act as a coherent receiver in that it is most responsive to coupled signals that are in phase with the forward power signal. The phase shifter 36 assures the proper phase coherence of the reflected power signal relative to the forward power signal for the mixer 30 to produce the maximum discernable mixer output. With the mixer forward power set to the appropriate level through the adjustable power 26, the output of the mixer 30 monotonically increases with an increase in the reflected coupled power. The mixer 30 demodulates or reduces to DC base band the value of the coupled power though the directional coupler 12. The DC output of the mixer 30 is filtered and amplified by amplifier 38 to produce a measurable output voltage that is related to the moisture content of the substrate placed across the gap 16. The amplifier 38 includes an adjustable gain 40 and an adjustable DC offset 42.

Referring now to FIGS. 4, 4A and 4B, use of the sensor 10 to determine the moisture content of hair will now be described in connection with a hair moisture sensor system 44. For example, hair moisture sensor system 44 may be used by a professional salon to quickly, accurately and reliably indicate to a stylist when the moisture content of a customer's hair is in the range of approximately 30-40% by weight so that the optimum styling results may then be achieved.

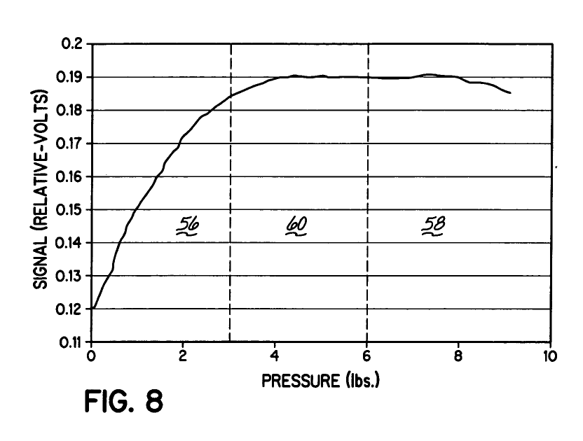

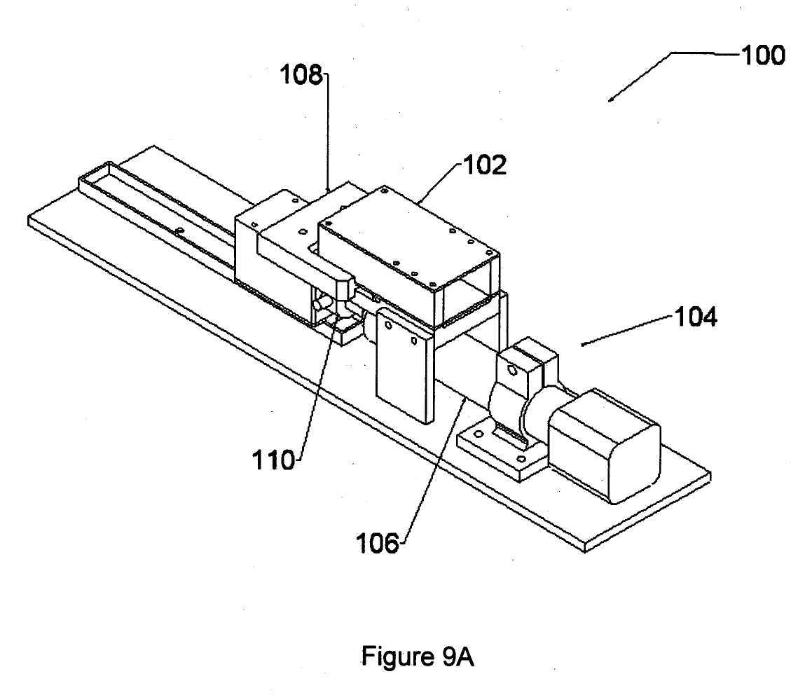

As shown in FIGS. 4A and 4B, a hair clamping device 46 is provided having pivoted jaws 48 and 50 that each terminate in a handle 52 that may be easily grasped and manipulated by the stylist. The jaws 48 and 50 may be biased to an open position as shown in FIG. 4A so that a bundle of hair 54 is readily received between the jaws 48, 50 and is oriented with the hair fibers 54 extending across, i.e., generally normal to the longitudinal axis of, the coupling gap 16 of the directional coupler 12 which is supported by jaw 50. As shown in FIG. 8, it has been determined that the packing pressure of the hair 54 across the coupling gap 16 is important to ensure reliability in the moisture content measurement. With low packing density below about three (3) lbs., i.e., a packing density in the pressure region 56, the output voltage signal of the mixer 30 may be unstable due to insufficient packing density of the hair fibers 54 across the coupling gap 16. At higher packing pressures above about seven (7) lbs., i.e., a packing density in the pressure region 58, the output voltage signal of the mixer 30 begins to fluctuate as the hair fibers 54 will exhibit the result of difference in packing density across the coupling gap 16. At these higher pressures, excess moisture is also quickly expelled resulting in unreliable lower readings. Packing fibers in the pressure region 60 can provide an output voltage signal from the mixer 30 that is stable to produce reliable and repeatable measurements of the moisture content.

In accordance with another aspect of the present invention, as shown in FIGS. 1, 2D, 4A and 4B, a pressure sensor 62 incorporating a film pressure transducer 64, is supported by the jaw 48 in juxtaposition to the directional coupler sensor 12. The pressure transducer 64 is operable to generate an output voltage signal that varies with the packing pressure applied to the hair 54 placed across the coupling gap 16. As shown in FIGS. 1 and 2D, the output voltage signal from the pressure transducer 64 is amplified by amplifier 66 having an adjustable gain 68 and DC offset 70, and that amplified output voltage signal is either provided directly at the output of the pressure sensor 62 through jumper 72, or it is applied as an input to a comparator 74 through jumper 76. A trigger voltage corresponding to a desired trigger pressure is set as a reference voltage 77 to the comparator 74. The measurement of the moisture content is triggered upon the crossing of the pre-set pressure threshold 77. This ensures that the desired compactness of the hair fibers 54 placed across the coupling gap 16 is achieved to obtain accurate, reliable and repeatable results. It will be understood by those of ordinary skill in the art that packing consistency can be achieved by a mechanical system (not shown) as well without departing from the spirit and scope of the present invention.

With reference to FIGS. 1 and 4, the measured signal from the sensor 10, and the trigger signal or pressure signal from the pressure sensor 62, are electrically coupled through a cable 78 to a processing system 80, such as a conventional PC or laptop computer. The processing system 80 is operable to convert the measurement signal generated by the sensor 10 into a moisture content value that may be presented on the display 82 of the system 80. As described in detail above, the measurement signal is triggered in response to the trigger signal generated by the pressure sensor 62. One or multiple measurements signals may be taken in response to the trigger signal.

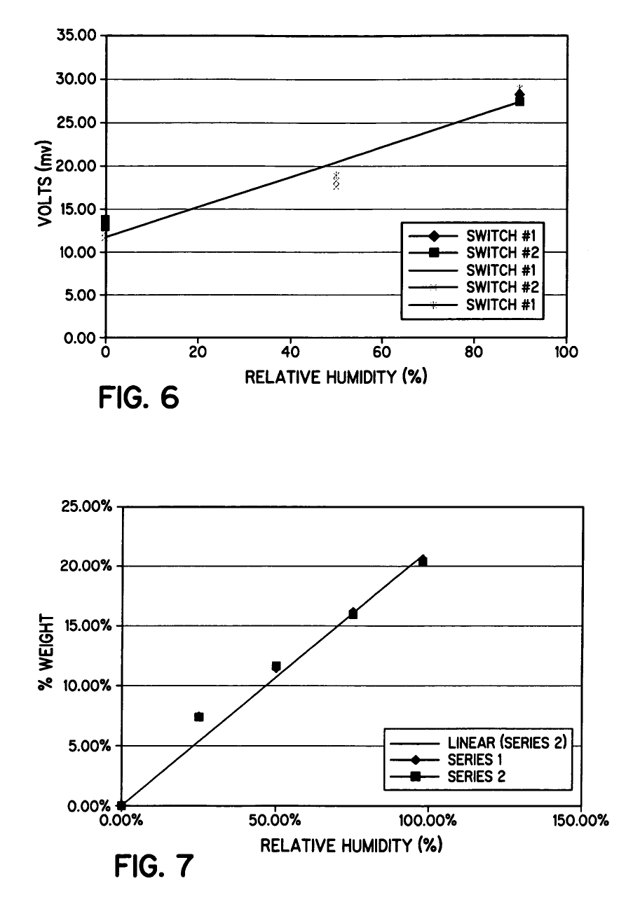

Referring now to FIGS. 6 and 7, the amplified output voltage of the sensor 10 is calibrated by first subjecting multiple switches of hair to a known moisture content via the use of relative humidity. The sensor 10 is then used to generate a measurement signal for each switch of hair at the various relative humidities, as shown in FIG. 6. Since hair exhibits a generally linear relationship between moisture content by weight and relative humidity as shown in FIG. 7, the processing system 80 is operable to convert the amplified output voltage of the sensor 10 into a value representing the moisture content by weight of the hair using a look-up table or algorithm. Since the water absorption and/or desorption capability of damaged hair and healthy hair will differ, the sensor 10 of the present invention may be used to provide a signal that is generally related to the health of the hair. Generally, the health of hair is characterized by such factors as smoothness, shine, absence of fragility, absence of fissuring, and absence of cuticular breakdown. As each of these factors is directly or indirectly related to the moisture content of the hair, the sensor 10 of the present invention is able to provide an accurate and reliable indication of the health of measured in vivo or in vitro hair.

The sensor 10 of the present invention provides a consumer friendly self-assessment tool that permits a consumer to periodically measure the general health of the consumer's hair. Based on these measurements, the consumer is able to take corrective actions as necessary which tend to improve the health of the consumer's hair. These actions may include changing hair care products, changing hair styling techniques, or both, so that the general health of the consumer's hair can be consistently monitored and improved. The sensor 10 also provides a useful monitoring tool to hair stylists and hair technicians as well.

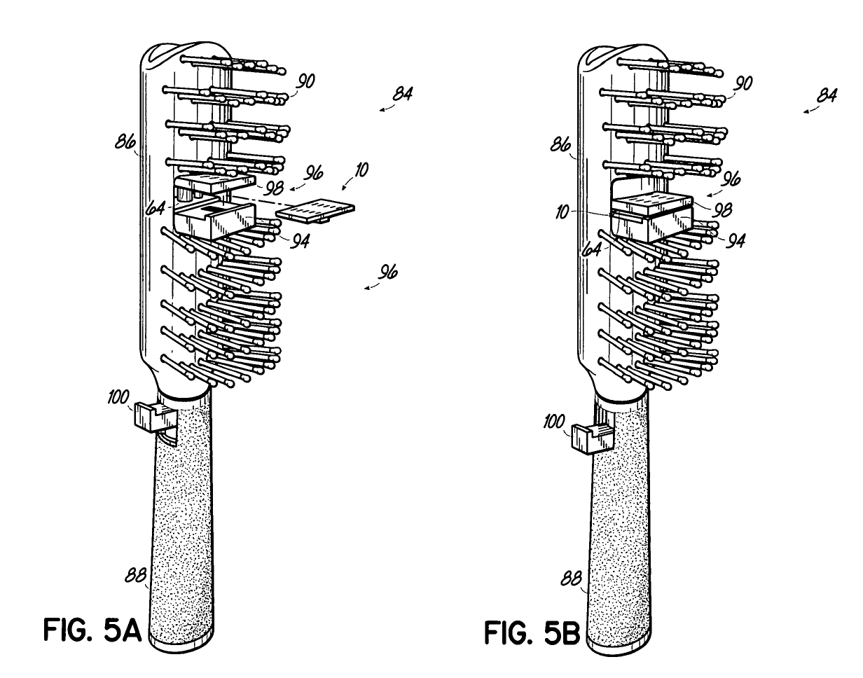

In accordance with another aspect of the present invention, as shown in FIGS. 5A and 5B, the sensor 10 is incorporated into a hair care product, such as a brush 84, used for grooming hair. The brush 84 includes an elongated body portion 86 terminating in a handle 88. Bristles 90 extend in a conventional manner from the body portion 86 of the brush 84 to enable grooming of the hair. In accordance with the principles of the present invention, as shown in FIG. 3, the signal generator 22, mixer 30, voltage regulator 92 (FIG. 2E) and electronics of the pressure sensor 62 are all integrated onto the PCB board 18 which is supported on a fixed base 94 of a hair clamping device 96 (FIGS. 5A and 5B). The fixed base 94 positions the directional coupler 12 near the bristles 90 so that measurements are easily taken while the hair is being brushed. The hair clamping device 96 includes a spring biased clamp member 98 that positions the pressure transducer 64 in juxtaposition to the directional coupler 12. A lever 100 is operatively connected to the movable clamp member 98 to enable a user to clamp hair across the coupling gap 16 when a sensor measurement is desired by moving the clamp member 98 toward the fixed base 94 as shown in FIG. 5B. The hair brush 84 may include LED's, and/or produce an audible signal, to provide an indication to the user about the moisture condition, health or other condition of the hair based on the sensor measurement. While not shown, it will be appreciated that the sensor 10 of the present invention may be incorporated into other hair care products as well, such as a comb, curling iron, or similar hair care product that preferably engages the user's hair during grooming to provide a measurement of the moisture content, health or other status of the hair based on the sensor measurement.

The directional coupler sensor 10 of the present invention is well suited to measure the moisture content, health or other condition of hair since it possesses sensitivity to variations in impedance in close proximity, such as about 0.1 in., to the surfaces of the strips 14a and 14b. The height of this effective measurement probing depth from the surfaces of the strips 14a, 14b is a function of the electromagnetic field that couples the strips 14a and 14b. The height of the measurement probing depth may be changed for a particular application by changing the height of the PCB 18, the dielectric constant of the PCB 18, the dimensions of the strips 14a, 14b, the coupling gap distance “s”, and/or the power supplied by the signal generator 22. By varying any or all of these parameters, the height of the coupling field can be altered to thereby change the effective measurement probing depth.

It is contemplated that sensor 10 may comprise multiple directional couplers 12 electrically coupled to at least one signal generator 22 to measure the respective moisture content of multiple substrates in accordance with the principles described in detail above. It is further contemplated that at least two of the multiple directional couplers 12 may have different effective measurement probing depths by varying one or more of the parameters described in detail above.

B. High Resonant, High-Q Circuit

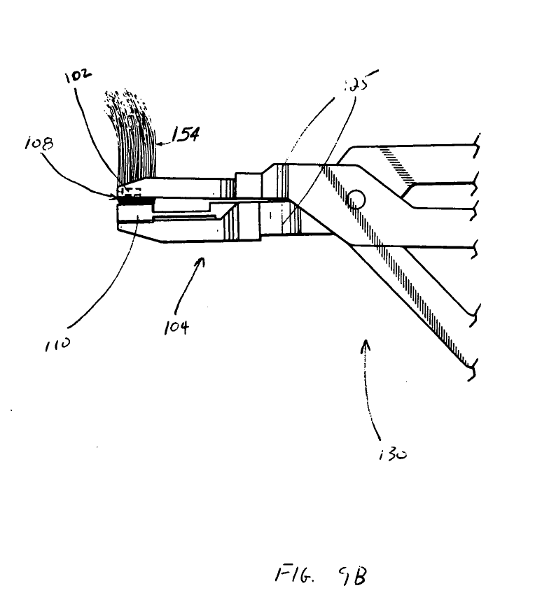

As shown in FIG. 9A, a high resonant, high-Q circuit 112 can be incorporated into a moisture measurement device 100 and used for the measurement of the moisture content of a substrate, such as a keratinous fiber, in accordance with the principles of the present invention. The moisture measurement device 100 incorporates a sensor 102 and a fiber matrix modification unit 104. Fiber matrix modification unit 104 generally comprises a linear actuator 106, and a packing area 108. Packing area 108 generally comprises a feedback mechanism 110 (such as a load cell). Sensor 102 generally comprises a high resonant, high-Q circuit 112 readily adaptable to analyze a wide variety of substrates, a wide variety of characteristics of these substrates (i.e., chemical and physical properties), and to measure different moisture related properties of those substrates as will be readily appreciated by those of ordinary skill in the art. Further, it will be appreciated by those of ordinary skill in the art that the present invention has use in a wide variety of applications and is therefore not limited only to the analysis of keratinous substrates or the measurement of only moisture content in hair.

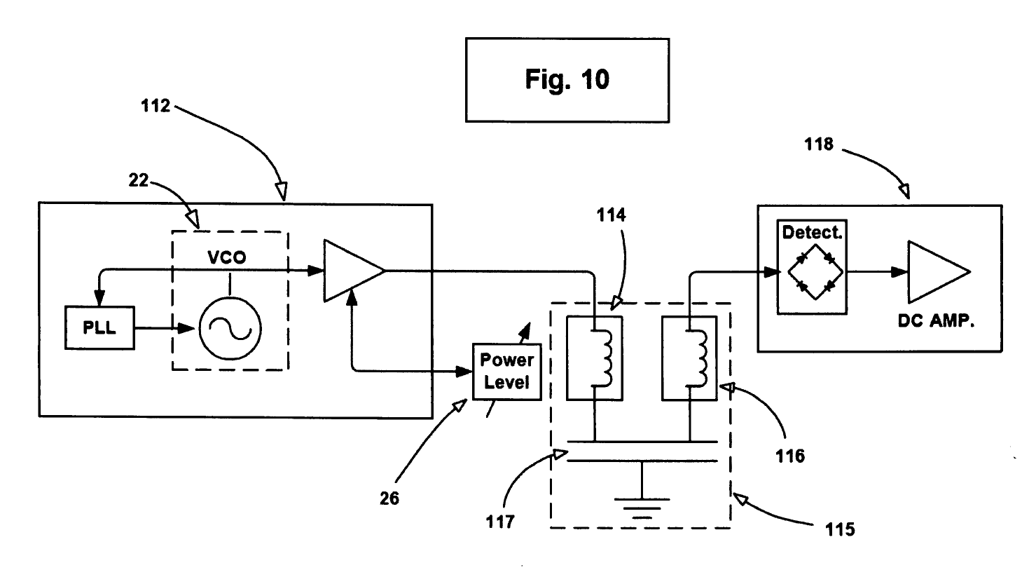

As shown in FIG. 10, high resonant, high-Q circuit 112 comprises a signal generator 122 to generate a power signal directly fed into a high-Q inductor 114. The signal generator 122 is phase locked to a crystal reference to maintain frequency and therefore measurement accuracy, stability, and repeatability. Signal generator 122 is also provided with an adjustable power level device 126. The signal generator 122 is preferably operable to generate signals in the VHF to UHF frequency ranges, i.e., between about 30 MHz and about 3 GHz, although other frequency ranges are possible as well. In accordance with one embodiment of the present invention, the signal generator 122 may operate at frequencies of about 1 GHz, such as frequencies ranging from about 860 MHz to about 928 MHz, and more preferably from about 865 MHz to about 915 MHz, and most preferably at about 915 MHz, since it is contemplated that the moisture (water) content of a substrate may be most accurately determined by its measured impedance in the near GHz range.

In accordance with one aspect of the present invention, the high resonant, high-Q circuit 112 is supplied with a fixed input frequency across the tuned LC circuit 115 comprising fixed-value inductors 114, 116 and capacitor 117. As a substrate is positioned proximate to capacitor 117, the value of capacitor 117 changes thereby causing a shift in the resonant frequency of high resonant, high-Q circuit 112. In a preferred embodiment, the substrate is placed proximate to the plates comprising capacitor 117. In a most preferred embodiment, the substrate is placed between the plates of capacitor 117 when capacitor 117 has a parallel plate configuration. AC/DC detector 118 then senses the resulting shift in the resonant frequency of high resonant, high-Q circuit 112. This shift can be plotted with respect to the phase locked crystal reference frequency to generate a resonance curve. AC/DC detector 118 can be provided with a DC amplifier, as would be known to one of skill in the art.

Without desiring to be bound by theory, it is believed that a substrate should be placed at least within the fringe fields of capacitor 117 for the determination of the moisture content of the substrate. The fringe fields of capacitor 117 are the lines of force generated by an electric field present between at least two conducting plates of capacitor 117. While it is believed that placement of the substrate proximate to the plates of capacitor 117 where the electric field is most intense will provide the most reproducible results for the determination of the moisture content of the substrate, one of skill in the art would be able to also determine the moisture content of a substrate placed within the fringe fields generated by the plates of capacitor 117 not proximate thereto. Additionally, one of skill in the art would also realize that any configuration of capacitor is suitable for use in the above-described LC circuit. This could include, but not be limited to, co-planar plate capacitors, non-parallel plate capacitors, inter-digitating plate capacitors, multiple plate capacitors, and combinations thereof.

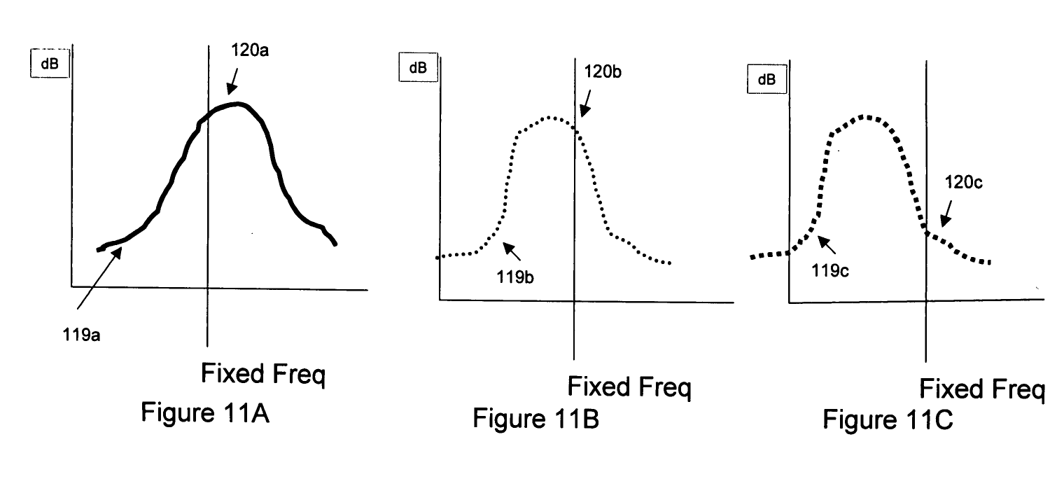

As shown in FIGS. 11A-11C, the resonance curves 119 of the high resonant, high-Q circuit 112 can be used to measure the moisture content of a substrate. In this regard, a fixed frequency inserted into LC circuit 115 can generate the exemplary resonance curve 119a, shown in FIG. 11A. Exemplary resonance curve 119a thereby shows an open-circuit value (i.e., no substance is present proximate to capacitor 117 wherein the output of AC/DC detector 118 provides a signal to the left of the resonant peak 120a. As shown in FIG. 11B, upon the introduction of a substrate containing less than 50 percent, preferably less than 10 percent, more preferably less than 1.0 percent, even more preferably less than 0.5 percent, and most preferably no moisture into LC circuit 115 proximate to capacitor 117, it can be observed that resonance curve 119b and resonant peak 120b shift to the left with respect to the fixed frequency input into LC circuit 115. This condition is generally referred to as the baseline condition. Upon the introduction of a saturated substrate into LC circuit 115 proximate to capacitor 117 (not shown), it can be observed that resonance curve 119c and resonant peak 120c shift further to the left with respect to the fixed frequency input into LC circuit 115 than was exhibited under the baseline condition. This state is shown in FIG. 11C. As would be known to one of skill in the art, the dynamic range of a signal processing system can be defined as the maximum dB level sustainable without overflow, or other distortion, (saturated condition) minus the dB level of the noise floor (baseline condition). The dynamic range of moisture measurement device 100 is determined by comparing the measured overall shift of resonance curve 119 and resonant peak 120 in transitioning from the baseline condition (FIG. 11B) to the saturated state (FIG. 11C). It would also be known to one of skill in the art that the introduction of a substrate proximate to the plates of a parallel plate capacitor 117 of LC circuit 115 can generate resonance curves 119 and resonant peaks 120 having shifts other than that described herein.

Referring again to FIG. 10, the measured signal from AC/DC detector 118 can be electrically coupled to a processing system, such as a conventional PC or laptop computer as would be known to one of skill in the art. The processing system can be operable to convert the output generated by the AC/DC detector 118 into a moisture content value that may be displayed (i.e., LED, CRT, LCD), or printed to a medium through devices known to those of skill in the art. Additionally, the processing system and display can be incorporated into a single apparatus (an integral device) suitable for use as a ‘stand-alone’ device for use in a laboratory, business, or clinical settings, or as a portable ‘hand-held’ apparatus suitable for use in the home or in travel situations. For example, moisture measurement device 100 comprising high resonant, high-Q circuit 112 may incorporated into a hand-held device that can be used by a professional salon to quickly, accurately and reliably indicate to a stylist when the moisture content of a customer's hair is in the appropriate range to achieve optimum styling or treatment results. Further, the output generated by the AC/DC detector 118 could be electrically coupled and stored in a memory device (i.e., EEPROM, flash memory cards, computer storage disks, or other data storage means known to those of skill in the art) integral to moisture measurement device 100, or communicated via modem, codec, USB, RS-232, wireless, and/or physical hard-wiring, and the like, to a remote computing system (nationally or internationally) for processing, storage, and/or display.

The amplified output voltage of the AC/DC detector 118 is calibrated by first subjecting multiple switches of hair to a known moisture content via the use of relative humidity. The AC/DC detector 118 is then used to generate a measurement signal for each switch of hair at the various relative humidities, as described supra. Since hair exhibits a generally linear relationship between moisture content by weight and relative humidity, discussed supra, the processing system can be operable to convert the amplified output voltage (signal) of the AC/DC detector 118 into a value representing the moisture content by weight of the hair using a look-up table or algorithm. As would be known to one of skill in the art, the look-up table or algorithm can contain signal reference values that are used for the comparison with the amplified output voltage of the AC/DC detector 118.

Referring again to the exemplary embodiment depicted in FIG. 9a, moisture measurement device 100 is provided with a fiber matrix modification unit 104 generally comprising a linear actuator 106, and a packing area 108. Packing area 108 generally comprises a feedback mechanism 110 (such as a load cell). Linear actuator 106 may be biased to an open position so that a fiber is readily received into packing area 108. Preferably, fibers are oriented to extend across, i.e., generally normal to the longitudinal axis of the packing area 108 of the fiber matrix modification unit 104. However, one of skill in the art will readily appreciate that the orientation of a fiber within packing area 108 does not explicitly require such alignment within packing area 108 because linear actuator 106 can control packing density. As discussed supra, it has been determined that the packing pressure of a substrate within packing area 108 is important to ensure reliability in the moisture content measurement. Additionally, providing packing area 108 with feedback mechanism 110 can ensure that the desired compactness of the substrate placed within packing area 108 achieves accurate, reliable and repeatable results. As would be known to one of skill in the art, exemplary, but non-limiting types of linear actuators 106 include electrical actuators, magnetic actuators, mechanical actuators, thermal actuators, and combinations thereof.

Referring to FIG. 9b, fiber matrix modification unit 104 can be embodied as a hand held device 130. In this regard, an exemplary substrate 154, such as a fiber, can be placed within the packing area 108 of the fiber matrix modification unit 104 of hand held device 130. The fiber matrix modification unit 104 can readily receive substrate 154 when feedback mechanism 110 (such as a load cell) is biased to an open position (jaws 125 of fiber matrix modification unit 104 of hand held device 130 being in an open position). It will be understood by those of ordinary skill in the art that packing consistency can be achieved by any system that incorporates a fiber matrix modification unit 104 without departing from the spirit and scope of the present invention. For example, fiber matrix modification unit 104 incorporating sensor 102 may be used by an end user, such as a professional salon to quickly, accurately and reliably indicate the moisture content of a customer's hair, or by a consumer to quickly, accurately and reliably indicate the moisture content of their hair at home. Additionally, one of skill in the art would be able to provide moisture measurement device 100 incorporating sensor 102 and fiber matrix modification unit 104 in a device capable of performing multiple measurements along the longitudinal axis of a substrate 154 or at any point on and/or within substrate 154.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A device for measuring the moisture content of a substrate, comprising:
a high-Q LC circuit having a resonant frequency, said LC circuit comprising a high-Q inductor and a capacitor; and,
a high frequency signal generator electrically coupled to said LC circuit, said high frequency signal generator being operable to couple power to said capacitor; and,
a fiber matrix modification unit; and,
wherein said resonant frequency of said LC circuit is changeable in response to said moisture content of said substrate when said substrate is placed within said fiber matrix modification unit and proximate to said capacitor.

2. The device of claim 1 wherein said fiber matrix modification unit comprises said capacitor.

3. The device of claim 2 wherein said capacitor comprises at least two plates and said substrate is placed proximate to said plates.

4. The device of claim 2 wherein said fiber matrix modification unit further comprises a linear actuator.

5. The device of claim 2 wherein said fiber matrix modification unit further comprises a packing area for placement of said substrate, said packing area further comprising a feedback mechanism.

6. The device of claim 1 wherein said high frequency generator high frequency signal generator is operable from about 30 MHz to about 3 GHz.

7. The device of claim 1 further comprising an AC/DC detector operably coupled to said high-Q LC circuit, said AC/DC detector being capable of sensing said change of said resonant frequency of said LC circuit.

8. The device of claim 7 wherein said AC/DC detector has an output, said output being electrically coupled to a system selected from the group consisting of processing systems, communications systems, display systems, storage systems, and combinations thereof.

9. The device of claim 8 wherein said system is integral with said device.

10. The device of claim 7 wherein said AC/DC detector has an output, said output being communicated to a system selected from the group consisting of processing systems, communications systems, display systems, storage systems, and combinations thereof.

11. The device of claim 1 wherein said substrate is a keratinous fiber.

12. The device of claim 1 wherein said fiber matrix modification unit comprises a fixed base member supporting said high-Q LC circuit and a movable member supporting said fiber matrix modification unit in juxtaposition to said high-Q LC circuit.

13. The device of claim 1 wherein said resonant frequency of said LC circuit provides a first frequency in response to a first substrate being placed within said fiber matrix modification unit, said first substrate having less than 50 percent moisture by weight of said substrate, and wherein said resonant frequency of said LC circuit provides a second frequency in response to a second substrate being placed within said fiber matrix modification unit, said second substrate being saturated.

14. The device of claim 13 wherein said device has a dynamic range, said dynamic range comprising a comparison of said first resonant frequency and said second resonant frequency.

15. A circuit for a device capable of measuring the moisture content of a substrate, said circuit comprising:
a high-Q LC circuit having a resonant frequency, said LC circuit comprising a high-Q inductor and a capacitor; and,
a high frequency signal generator electrically coupled to said LC circuit and operable to couple power to said capacitor; and,
wherein said resonant frequency of said LC circuit is changeable in response to said-moisture content of said substrate placed proximate to said capacitor.

16. The circuit of claim 15 further comprising an AC/DC detector, said AC/DC detector being operably coupled to said circuit, said AC/DC detector being capable of detecting said change of said resonant frequency of said LC circuit.

17. The circuit of claim 16 wherein said AC/DC detector further comprises a DC amplifier.

18. The circuit of claim 15 wherein said circuit has a shiftable resonance curve.

19. The circuit of claim 15 wherein said high frequency generator high frequency signal generator is operable from about 30 MHz to about 3 GHz.

20. A method for measuring the moisture content of a substrate, said method comprising the steps of:
(a) providing a high-Q LC circuit having a shiftable resonance curve, said LC circuit comprising a high-Q inductor and a capacitor, and a high frequency signal generator electrically coupled to said LC circuit, said high frequency signal generator being operable to couple power to said capacitor;
(b) introducing said substrate proximate to said capacitor;
(c) measuring an output of said high-Q LC circuit; and,
(d) comparing said output with a reference to determine said moisture content of said substrate.
